(19) United States
(12) Patent Application Publication
(10) Pub. No.: US 2009/0117079 A1
(43) Pub. Date: May 7, 2009

(54) METHODS AND MATERIALS FOR TREATING OR PREVENTING DISEASES/DISORDERS MEDIATED BY ALCOHOL DEHYDROGENASE

(76) Inventor: Woodrow C. Monte, Page, AZ (US)

Correspondence Address: K&L Gates LLP, P.O. Box 1135, CHICAGO, IL, 60690, US

(21) Appl. No.: 12/260,777

(22) Filed: Oct. 29, 2008

Related U.S. Application Data
(60) Provisional application No. 60/985,054, filed on Nov. 2, 2007.

(51) Int. Cl. A61K 45/00; A61K 31/045; A61P 37/02; A61K 31/415

(52) U.S. Cl. 424/93.1; 514/724; 514/406

(57) ABSTRACT

The present disclosure relates generally to methods and materials for treating and/or preventing a disease or disorder in a subject that is mediated by alcohol dehydrogenase by administering to the subject a therapeutically effective amount of one or more agents that prevents and/or inhibits the metabolism of methanol by alcohol dehydrogenase. Methanol metabolism may be reduced/inhibited in a subject by administering to the subject a therapeutically effective amount of ethanol.

### FIELD

The present disclosure relates generally to methods and materials for treating or preventing diseases/disorders (e.g., a cancer, an immune system disease or a neurodegenerative disease, including for example Alzheimer's disease) mediated by alcohol dehydrogenase in a subject by administering to the subject a therapeutically effective amount of an agent capable of inhibiting the metabolism of methanol. These methods may comprise administering ethanol in a therapeutically effective amount to a subject to reduce/inhibit methanol metabolism.

### BACKGROUND OF THE INVENTION

Alcohol dehydrogenase (ADH I) is an enzyme found primarily in certain epithelial tissues of the body and lining of the stomach and liver where it helps break down alcohol into substances that can be excreted from the body. There are five classes (I-V) of alcohol dehydrogenase with class 1 the primary form responsible for conversion of alcohols to aldehydes in humans. Class 1 ADH (ADH I) consists of A, B, and C subunits that are encoded by the genes ADH1A, ADH1B, and ADH1C. ADH I catalyzes the oxidation of ethanol to acetaldehyde:

H₃CH₂OH+NAD⁺→CH₃CHO+NADH+H⁺

The enzymatic activity of ADH I permits the consumption of alcoholic beverages, but its evolutionary purpose is poorly understood and likely goes beyond the breakdown of alcohols naturally contained in foods or produced by bacteria in the digestive tract. Specifically, ADH I converts ethanol into acetaldehyde, which is then converted to carbon dioxide and water. ADH I is also involved in the toxicity of other types of alcohol: for instance, it oxidizes methanol to produce notably formaldehyde and ethylene glycol to ultimately yield glycolic and oxalic acids. Formaldehyde is a well known cross-linking agent that can inactivate, stabilize, or immobilize proteins.

Numerous diseases or disorders are associated with the inactivation of a protein. For example, in Alzheimer's disease Tau protein is misfolded and polymerized producing neurofibrillary tangles in the brain and in some cancers proteins are inactivated that are responsible for the control of cellular division. In autoimmune disease, proteins normally recognized as “self” become immunogenic by chemical, physical or biological alteration. Strategies employed for the treatment of these diseases generally seek to manage the symptoms associated with the disease. Given that these treatments generally do not target the etiology of the disease, there exists a need to develop strategies for treating and/or preventing the origin of these diseases.

### SUMMARY

The present disclosure relates generally to methods and materials for the treatment and/or prevention of diseases or disorders mediated by alcohol dehydrogenase (ADH) (e.g., class 1 alcohol dehydrogenase).

Methods are provided for treating and/or preventing diseases or disorders mediated by alcohol dehydrogenase in a subject (e.g., human) by administering to the subject a therapeutically effective amount of one or more agents that inhibit the metabolism of methanol.

Methods are also provided for the prevention of a disease or disorder mediated by alcohol dehydrogenase in a subject by administering to the subject a therapeutically effective amount of an agent that inhibits the metabolism of methanol.

In some embodiments, the agent may inhibit the metabolism of methanol to formaldehyde and formal hydrate. In further embodiments, the agent is ethanol or 4-methylpyrazole (Fomepizole).

In other embodiments, the agent is a microorganism that is capable of converting dietary carbohydrates to ethanol. In further embodiments, the agent is a microorganism that is capable of producing ethanol through the microorganism's own metabolic processes.

In some embodiments, the disease or disorder is cancer. Cancers may include, but are not limited to breast cancer, adenocarcinoma, melanoma, glioblastoma and mononuclear cell leukemia.

In other embodiments, the disease or disorder is an autoimmune disease. In some embodiments, the autoimmune disease is a Th1-mediated disease. In further embodiments, the Th1-mediated autoimmune disease is a demyelinating disease. In yet further embodiments, the demyelinating disease is multiple sclerosis. Autoimmune diseases may include Alzheimer's disease, multiple sclerosis, atherosclerosis, rheumatoid arthritis and lupus erythematosus.

In other embodiments, the disease or disorder may include neural tube birth defects, fetal alcohol syndrome or autism.

In some embodiments, the agent is administered by one or more of the following routes: intramuscular injection, subcutaneous injection, intravenous administration, parenteral, intranasal, intrapulmonary and oral.

In some embodiments, the subject has been diagnosed with cancer. In further embodiments, the subject has been diagnosed with an immune system disease (e.g., autoimmune disease). In other embodiments, the subject is at risk of developing an immune system disease. In further embodiments, the subject is a human.

### BRIEF DESCRIPTION OF THE DRAWINGS

FIG. 1 depicts the metabolism of methanol and ethanol by alcohol dehydrogenase (ADH I). In humans, low concentrations of ethanol in the blood acts by competitive inhibition of ADH I (10/1 ethanol/methanol affinity ratio) to stop methanol from converting to formaldehyde in the target organs. Ethanol and 4-methylpyrazole (Fomepizole), under circumstances where methanol cannot be removed from the environment, acts as protective substances which prevent the production of formaldehyde inside sensitive organs. In the absence of ethanol or 4-methylpyrazole, methanol may be converted to formaldehyde which may lead to cancer, an immune system disease or a neurodegenerative disease, including, for example Alzheimer's disease.

### DETAILED DESCRIPTION

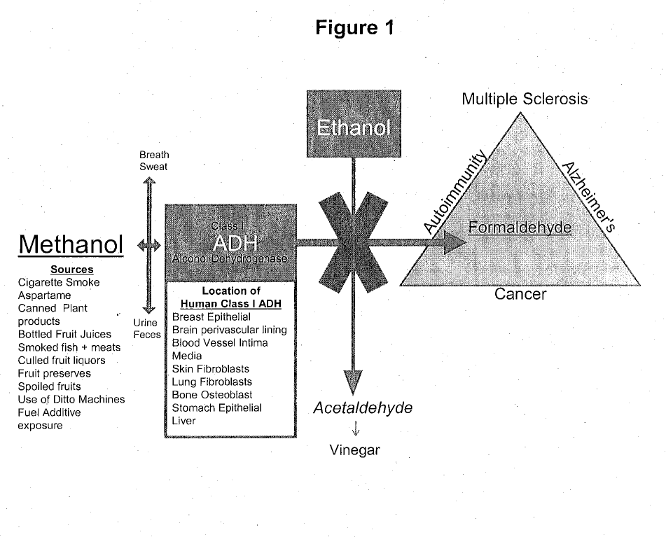

The present disclosure provides methods for the treatment and/or prevention of one or more diseases and/or disorders mediated by alcohol dehydrogenase activity. The metabolism of methanol produces both formaldehyde and formal hydrate, both of which may react with a protein thereby creating a modified protein. These modified proteins may be recognized by the immune system as foreign or their native function may be adversely affected such as, for example, with Alzheimer's disease. Surprisingly, it has been discovered that the metabolism of methanol by alcohol dehydrogenase class I leads to disease (e.g., an ADH I mediated disease or disorder) (see, e.g., FIG. 1). It is hypothesized that alcohol dehydrogenase present in epithelial tissue encounters methanol as it leaves the bloodstream where it is metabolized to formaldehyde and formal hydrate. The formaldehyde and formal hydrate may diffuse into nearby tissue, where they may react with and modify protein. For example, it is believed that proteins modified by formaldehyde may be recognized by macrophages, which may mount an immune response to the modified protein. Additionally or alternatively, the modified proteins may be inactivated leading to a cancer (e.g., breast cancer) or a neurodegenerative disease, including for example Alzheimer's disease. As such, the present disclosure provides methods for the treatment and/or prevention of one or more diseases or disorders mediated by the activity of alcohol dehydrogenase by administering an agent to a subject that inhibits the metabolism of methanol to formaldehyde and formal hydrate.

Methods are provided for the treatment of one or more diseases and/or disorders (e.g., a cancer, an immune system disease or a neurodegenerative disease including for example Alzheimer's disease) in a subject mediated by alcohol dehydrogenase by administering to the subject a therapeutically effective amount of ethanol, wherein the ethanol inhibits or reduces the metabolism of methanol to formaldehyde and formal hydrate (e.g., by alcohol dehydrogenase).

Methods are also provided for the treatment of one or more diseases and/or disorders in a subject mediated by alcohol dehydrogenase by administering to the subject a therapeutically effective amount of a microorganism that is capable of converting dietary carbohydrates to ethanol, wherein the ethanol inhibits or reduces the metabolism of methanol to formaldehyde and formal hydrate.

Methods are provided for the treatment of one or more diseases and/or disorders in a subject mediated by alcohol dehydrogenase by administering to the subject a therapeutically effective amount of a microorganism that is capable of producing ethanol through the microorganism's own metabolic processes, wherein the ethanol inhibits or reduces the metabolism of methanol to formaldehyde and formal hydrate.

Methods are provided for the treatment of one or more diseases and/or disorders in a subject mediated by alcohol dehydrogenase by administering to the subject a therapeutically effective amount of ethanol, wherein the ethanol inhibits or reduces the metabolism of methanol to formaldehyde and formal hydrate.

Methods are also provided for the treatment of one or more disease and/or disorders in a subject mediated by alcohol dehydrogenase by administering to the subject a therapeutically effective amount of a microorganism that is capable of converting dietary carbohydrates to ethanol, wherein the ethanol inhibits or reduces the metabolism of methanol to formaldehyde and formal hydrate.

Methods are provided for the treatment of one or more disease and/or disorders in a subject mediated by alcohol dehydrogenase by administering to the subject a therapeutically effective amount of a microorganism that is capable of producing ethanol through the microorganism's own metabolic processes, wherein the ethanol inhibits or reduces the metabolism of methanol to formaldehyde and formal hydrate.

Methods are provided for the treatment of an immune system disease, including, for example, an autoimmune disease (e.g., Alzheimer's disease or multiple sclerosis) in a subject by administering to the subject a therapeutically effective amount of ethanol, wherein the ethanol inhibits or reduces the metabolism of methanol to formaldehyde and formal hydrate.

Methods are also provided for the treatment of an immune system disease, including, for example, an autoimmune disease (e.g., Rheumatoid arthritis or multiple sclerosis) in a subject by administering to the subject a therapeutically effective amount of a microorganism that is capable of converting dietary carbohydrates to ethanol, wherein the ethanol inhibits or reduces the metabolism of methanol to formaldehyde and formal hydrate.

Methods are provided for the treatment of an immune system disease, including, for example, an autoimmune disease (e.g., Rheumatoid arthritis or multiple sclerosis) in a subject by administering to the subject a therapeutically effective amount of a microorganism that is capable of producing ethanol through the microorganism's own metabolic processes, wherein the ethanol inhibits or reduces the metabolism of methanol to formaldehyde and formal hydrate.

Methods are provided for the prevention of an immune system disease, including, for example, an autoimmune disease in a subject (e.g., a subject at risk for developing an autoimmune disease) by administering to the subject a therapeutically effective amount of ethanol, wherein the ethanol inhibits or reduces the metabolism of methanol to formaldehyde and formal hydrate.

Methods are also provided for the prevention of an immune system disease, including, for example, an autoimmune disease in a subject by administering to the subject a therapeutically effective amount of a microorganism that is capable of converting dietary carbohydrates to ethanol, wherein the ethanol inhibits or reduces the metabolism of methanol to formaldehyde and formal hydrate.

Methods are provided for the prevention of an immune system disease, including, for example, an autoimmune disease in a subject by administering to the subject a therapeutically effective amount of a microorganism that is capable of producing ethanol through the microorganism's own metabolic processes, wherein the ethanol inhibits or reduces the metabolism of methanol to formaldehyde and formal hydrate.

Autoimmune diseases that may be treated or prevented by the presently disclosed methods may include Alzheimer's disease, multiple sclerosis, atherosclerosis, rheumatoid arthritis and lupus erythematosus.

Methods are provided for the prevention of a cancer or neurodegenerative disease, including for example Alzheimer's disease in a subject (e.g., a subject at risk for developing a cancer) by administering to the subject a therapeutically effective amount of ethanol, wherein the ethanol inhibits or reduces the metabolism of methanol to formaldehyde and formal hydrate.

Methods are also provided for the prevention of a cancer or neurodegenerative disease, including for example Alzheimer's disease in a subject by administering to the subject a therapeutically effective amount of a microorganism that is capable of converting dietary carbohydrates to ethanol, wherein the ethanol inhibits or reduces the metabolism of methanol to formaldehyde and formal hydrate.

Methods are provided for the prevention of a cancer or neurodegenerative disease, including for example Alzheimer's disease in a subject by administering to the subject a therapeutically effective amount of a microorganism that is capable of producing ethanol through the microorganism's own metabolic processes, wherein the ethanol inhibits or reduces the metabolism of methanol to formaldehyde and formal hydrate.

Cancers that may be prevented by the presently disclosed methods include breast cancer, adenocarcinomas, melanoma, glioblastoma mononuclear cell leukemia.

Agents that inhibit or reduce the metabolism of methanol to formaldehyde and formal hydrate may be administered by one or more of the following routes: intramuscular injection, subcutaneous injection, intravenous administration, parenteral, intranasal, intrapulmonary and oral.

Methods for Treating or Preventing Diseases and/or Disorders Mediated by Alcohol Dehydrogenase

Methods are provided for treating one or more diseases or disorders in a subject that are mediated by alcohol dehydrogenase activity. The methods may comprise administering to the subject a therapeutically effective amount of one or more agents that inhibit the metabolism of methanol by alcohol dehydrogenase. For example, a subject may be treated for an immune system disease, a cancer or neurodegenerative disease, including for example Alzheimer's disease by administering to the subject a therapeutically effective amount of an agent capable of inhibiting the metabolism of methanol. As used herein, the term “treating” a disease means to manage a disease by medicinal or other therapies. Treatment of a disease may ameliorate the symptoms of a disease, reduce the severity of a disease, alter the course of disease progression and/or ameliorate or cure the basic disease problem.

Methods are also provided for preventing one or more diseases or disorders in a subject that are mediated by alcohol dehydrogenase activity. For example, a subject may be prevented from developing an immune system disease, a cancer or a neurodegenerative disease, including for example Alzheimer's disease by administering to the subject a therapeutically effective amount of an agent capable of inhibiting the metabolism of methanol. As used herein, the term “preventing a disease” means inhibiting or reversing the onset of the disease, inhibiting or reversing the initial signs of the disease or inhibiting the appearance of clinical symptoms of the disease.

As used herein, the term “therapeutically effective amount” refers to the amount of agent (e.g., ethanol) effective to prevent a subject from metabolizing methanol. For example, when ethanol is used as an agent to treat or prevent one or more diseases or disorders mediated by alcohol dehydrogenase, it is preferred that a carrier means release ethanol into the digestive or respiratory tract in a subject at a rate sufficient to maintain a concentration of ethanol in the blood stream generally in the range of one to two hundred and fifty parts per million, more preferably, ten to fifty parts per million (ppm).

Immune system diseases that may be treated and/or prevented according to the methods disclosed herein include, e.g., acute immune diseases, chronic immune diseases and autoimmune diseases. As used herein, “immune system disease” means any disease mediated by T-cell interactions with B7-positive cells including, but not limited to, autoimmune diseases and immunoproliferative diseases. In some embodiments, the immune system disease is an autoimmune disease including, for example, a Th1-mediated disease such as a demyelinating disease (e.g., multiple sclerosis). Autoimmune diseases may include, but are not limited to, Alzheimer's disease, multiple sclerosis, atherosclerosis, rheumatoid arthritis and lupus erythematosus.

Cancers that may be treated and/or prevented according to the methods of the present disclosure, include, but are not limited to breast cancer, adenocarcinoma, melanoma, glioblastoma and mononuclear cell leukemia.

The invention also includes methods for treating a subject at risk for developing a disease or disorder mediated by alcohol dehydrogenase (e.g., a cancer, an immune system disease or a neurodegenerative disease, including for example Alzheimer's disease). For example, a subject at risk for developing an autoimmune disease or cancer or Alzheimer's disease may be treated by administering to the subject a therapeutically effective amount of an agent capable of inhibiting the metabolism of methanol. Being at risk for the disease can result from, e.g., a family history of the disease, a genotype which predisposes to the disease, or phenotypic symptoms which predispose to the disease.

In some embodiments, the therapeutically effective amount of an alcohol (e.g., ethanol) may be administered to the subject. For example, a carrier means may be selected to permit the gradual release of ethanol into the digestive or respiratory tract in minor effective amounts sufficient to inhibit the metabolism of methanol in the body and sufficient to avoid the intoxication of the individual by the ethanol.

Sources of ethanol include, for example, ethyl alcohol, ethyl esters of various chemicals (e.g., pectin ethylester) and microorganisms that can survive within a dialytic capsule or other ethanol source carrier means and either convert through fermentation various dietary carbohydrates into ethanol or otherwise produce ethanol through the microorganisms' own metabolic processes.

The source of ethanol may be administered into the digestive tract from within carrier means comprising a capsule having a dialytic wall with pores which permit ethanol carried in the capsule to slowly bleed through the pores into the digestive tract. The ethanol may be mixed with propylene glycol, glycerol, water, potassium or sodium stearate or other solid or liquid substances which function as carrier means to slow or increase the rate of diffusion of ethanol through the dialytic wall of the capsule. The capsule can be orally, suppositorally, or surgically introduced into the digestive tract. The dialytic wall of the capsule presently preferably comprises a cellophane or polycarbonate film having pores approximately 10 Angstroms in diameter. The width of each pore opening can vary, but is preferably in the range of 5 to 30 Angstroms. The dialytic film or wall is preferably resistant to degradation by the digestive system for a period of time necessary to permit all or nearly all of the ethanol carried within the dialytic film to gradually bleed through the pores into the digestive tract.

When the carrier capsule or tablet is administered orally, it may be desirable that it be coated with an enteric substance so the capsule will not be destroyed prior to its reaching the lower intestinal tract. If a capsule or tablet having a dialytic film is not utilized and a source of ethanol is simply mixed or chemically combined with a carrier substance which slows the release of ethanol in the intestinal tract, it may be preferred that the ethanol source-carrier substance mixture be resistant to stomach digestive chemicals so the ethanol will not be completely released in the stomach.

### Pharmaceutical Formulations

Pharmaceutical formulations comprising an agent (e.g., ethanol) capable of inhibiting the metabolism of methanol are provided. Such formulations may be prepared for storage by mixing an agent having the desired degree of purity with optional pharmaceutically acceptable carriers, excipients, or stabilizers (Remington's Pharmaceutical Sciences 16th edition, Osol, A. Ed. (1980)), in the form of lyophilized formulations or aqueous solutions.

Acceptable carriers, excipients, or stabilizers are nontoxic to subjects at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; catechol; resorcinol; cyclohexanol; 3-pentanol; and m-cresol); low-molecular-weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Preferred lyophilized formulations are described in WO 1997/04801, expressly incorporated herein by reference.

The formulations to be used for in vivo administration must be sterile. This may be accomplished by filtration through sterile filtration membranes.

The formulation herein may also contain more than one active compound as necessary for the particular indication being treated, preferably those with complementary activities that do not adversely affect each other. Alternatively, or additionally, the composition may further comprise a chemotherapeutic agent, cytotoxic agent, cytokine, growth-inhibitory agent, anti-hormonal agent, anti-angiogenic agent, and/or cardioprotectant. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug-delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the agent (e.g., ethanol), which matrices are in the form of shaped articles, e.g. films, or microcapsules. Examples of sustained-release matrices include polyesters, hydrogels (e.g., poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPO® (injectable microspheres composed of lactic acid-glycolic acid copolymer and leuprolide acetate), and poly-D-(−)-3-hydroxybutyric acid.

The pharmaceutical composition of the invention may permit the gradual controlled release into the blood stream of minor effective amounts of one or more agents (e.g., ethanol) to prevent the metabolism of methanol and which, at the same time, are not either sufficient to cause intoxication of an individual or sufficient to prevent methanol from being removed by the body through the breath and urine. The composition can be readily ingested by an individual without requiring the utilization of intravenous or other medical equipment.

Pharmaceutical formulations comprising an agent (e.g., ethanol or 4-methylpyrazole) capable of inhibiting the metabolism of methanol may be administered to the subject as often as necessary to keep the concentration of agent in the blood stream at a therapeutically effective amount.

In some embodiments, it may be desirable to achieve a rapid release of the agent (e.g., ethanol). For example, the wall of the capsule or carrier means can be selected such that it may be rapidly destroyed by the digestive system (e.g., the capsule wall or coating enclosing the agent may be comprised of a gelatin). In some embodiments, it may be desirable to achieve a slow release of the agent. For example, an agent may be mixed or chemically combined or bound with a thickener or some other carrier composition which would slow its release in the digestive tract.

The membrane wall of film encapsulating a source of ethanol may be comprised of a physiologically nontoxic material. Since ethanol may be volatile and evaporate rapidly when exposed to the atmosphere, capsules carrying ethanol may be coated with a material which prevents or slows evaporation of ethanol into the atmosphere. Alternatively, the capsules may be stored in a container which maintains a vapor pressure equal to or greater than that of ethanol, or, may be stored in a solution which has equivalent ethanol content and which does not break down or alter the chemical composition of the ethanol source and its carrier means.

### Articles of Manufacture

Articles of manufacture containing materials useful for the treatment of diseases or disorders mediated by alcohol dehydrogenase are provided. The article of manufacture may comprise a container and a label or package insert on or associated with the container. Suitable containers include, for example, bottles, vials or syringes. The containers may be formed from a variety of materials such as glass or plastic. The container holds a composition that may be effective for treating the condition and may have a sterile access port (e.g., the container may be an intravenous solution bag or a vial having a stopper pierceable by a hypodermic injection needle). At least one active agent in the composition may be an agent capable of inhibiting the metabolism of methanol. The label or package insert may indicate that the composition may be used for treating the condition of choice, such as an immune system disease and/or cancer and/or a neurodegenerative disease, including for example Alzheimer's disease. In one embodiment, the label or package insert may indicate that the composition comprising the agent capable of inhibiting the metabolism of methanol may be used to treat a Th1-mediated disease (e.g., a demyelinating disease, such as multiple sclerosis).

Moreover, the article of manufacture may comprise (a) a first container with a composition contained therein, wherein the composition comprises an agent capable of inhibiting the metabolism of methanol, and (b) a second container with a composition contained therein, wherein the composition comprises a therapeutic agent other than the agent capable of inhibiting the metabolism of methanol. The article of manufacture in this embodiment of the disclosure may further comprise a package insert indicating that the first and second compositions can be used in combination to treat an autoimmune disease. Such therapeutic agents may be any of the adjunct therapies described in the preceding section (e.g., a thrombolytic agent, an anti-platelet agent, a chemotherapeutic agent, an anti-angiogenic agent, an anti-hormonal compound, a cardioprotectant, and/or a regulator of immune function in a mammal, including a cytokine). Alternatively, or additionally, the article of manufacture may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

While the present disclosure has been described and illustrated herein by references to various specific materials, procedures and examples, it is understood that the disclosure is not restricted to the particular combinations of material and procedures selected for that purpose. Numerous variations of such details can be implied as will be appreciated by those skilled in the art. It is intended that the specification be considered as exemplary, only, with the true scope and spirit of the disclosure being indicated by the following claims. All references, patents, and patent applications referred to in this application are herein incorporated by reference in their entirety.

1. A method for the treatment of a disease or disorder mediated by alcohol dehydrogenase in a subject, the method comprising: administering to the subject a therapeutically effective amount of one or more agents that inhibit the metabolism of methanol by alcohol dehydrogenase.

2. The method of claim 1, wherein the agent inhibits the metabolism of methanol to formaldehyde and formal hydrate.

3. The method of claim 1, wherein the agent is ethanol or 4-methylpyrazole.

4. The method of claim 1, wherein the agent is a microorganism that is capable of converting dietary carbohydrates to ethanol.

5. The method of claim 1, wherein the agent is a microorganism that is capable of producing ethanol through the microorganism's own metabolic processes.

6. The method of claim 1, wherein the disease or disorder is an autoimmune disease.

7. The method of claim 6, wherein the autoimmune disease is a Th1-mediated disease.

8. The method of claim 7, wherein the Th1-mediated autoimmune disease is a demyelinating disease.

9. The method of claim 8, wherein the demyelinating disease is multiple sclerosis.

10. The method of claim 6, wherein the autoimmune disease is selected from the group consisting of Alzheimer's disease, multiple sclerosis, atherosclerosis, rheumatoid arthritis and lupus erythematosus.

11. The method of claim 1, wherein the disease or disorder is cancer or Alzheimer's disease.

12. The method of claim 11, wherein the cancer is a carcinoma, lymphoma, blastoma, sarcoma or leukemia.

13. The method of claim 11, wherein the cancer is selected from the group consisting of: breast cancer, adenocarcinoma, melanoma, glioblastoma and mononuclear cell leukemia.

14. The method of claim 1, wherein the agent is administered by a route selected from the group consisting of intramuscular injection, subcutaneous injection, intravenous administration, parenteral, intranasal, intrapulmonary and oral.

15. The method of claim 1, wherein the subject has been diagnosed with an autoimmune disease.

16. The method of claim 1, wherein the subject is a human.

17. A method for the prevention of a disease or disorder mediated by alcohol dehydrogenase in a subject, the method comprising: administering to the subject a therapeutically effective amount of one or more agents that inhibit the metabolism of methanol by alcohol dehydrogenase.

18. The method of claim 17, wherein the agent inhibits the metabolism of methanol to formaldehyde and formal hydrate.

19. The method of claim 17, wherein the agent is ethanol or 4-methylpyrazole.

20. The method of claim 17, wherein the agent is a microorganism that is capable of converting dietary carbohydrates to ethanol.

21. The method of claim 17, wherein the agent is a microorganism that is capable of producing ethanol through the microorganism's own metabolic processes.

22. The method of claim 17, wherein the disease or disorder is an autoimmune disease.

23. The method of claim 22, wherein the autoimmune disease is a Th1-mediated disease.

24. The method of claim 23, wherein the Th1-mediated autoimmune disease is a demyelinating disease.

25. The method of claim 24, wherein the demyelinating disease is multiple sclerosis.

26. The method of claim 22, wherein the autoimmune disease is selected from the group consisting of Alzheimer's disease, multiple sclerosis, atherosclerosis, rheumatoid arthritis and lupus erythematosus.

27. The method of claim 17, wherein the disease or disorder is cancer.

28. The method of claim 27, wherein the cancer is a carcinoma, lymphoma, blastoma, sarcoma or leukemia.

29. The method of claim 27, wherein the cancer is selected from the group consisting of: breast cancer, adenocarcinoma, melanoma, glioblastoma and mononuclear cell leukemia.

30. The method of claim 17, wherein the agent is administered by a route selected from the group consisting of intramuscular injection, subcutaneous injection, intravenous administration, parenteral, intranasal, intrapulmonary and oral.

31. The method of claim 17, wherein the subject is at risk of developing an immune system disease.

32. The method of claim 17, wherein the subject is at risk of developing cancer.

33. The method of claim 17, wherein the subject is a human.
